(19) United States
(12) Patent Application Publication
(10) Pub. No.: US 2023/0377732 A1
(43) Pub. Date: Nov. 23, 2023

(54) SYSTEMS AND METHODS FOR INCIDENT AND EVENT REPORTING

(71) Applicant: Compliance Partners LLC, New Orleans, LA (US)

(72) Inventors: Heidi Raines, New Orleans, LA (US); Jessica Smith, New Orleans, LA (US); Brandon Bergeron, New Orleans, LA (US)

(21) Appl. No.: 18/310,871

(22) Filed: May 2, 2023

Related U.S. Application Data
(60) Provisional application No. 63/343,310, filed on May 18, 2022.

(51) Int. Cl. G16H 40/20

(52) U.S. Cl. CPC G16H 40/20

(57) ABSTRACT

Embodiments of the present disclosure are directed to healthcare incident and event reporting and management. According to one embodiment, healthcare incident and event reporting and management can be implemented as a cloud-based service and provided to clients, such as healthcare providers, for example, over the Internet and/or other communications networks. Generally speaking, incident and event reporting and management can include reporting, follow-up management, and post-event analysis to help healthcare providers control and reduce the number of harmful incidents and events.

### CROSS REFERENCE TO RELATED APPLICATION

The present application claims the benefits of and priority, under 35 U.S.C. § 119(e), to U.S. Provisional Application No. 63/343,310 filed May 18, 2022 by Raines, et. al. and entitled “Systems and Methods for Incident and Event Reporting” of which the entire disclosure is incorporated herein by reference for all purposes.

### FIELD OF THE DISCLOSURE

Embodiments of the present disclosure relate generally to methods and systems for managing electronic records related to healthcare and more particularly to receiving and processing electronic records defining reports of incidents or events related to delivery of healthcare services.

### BRIEF DESCRIPTION OF THE DRAWINGS

FIG. 1 is a block diagram illustrating elements of an exemplary computing environment in which embodiments of the present disclosure may be implemented.

FIG. 2 is a block diagram illustrating elements of an exemplary computing device in which embodiments of the present disclosure may be implemented.

FIG. 3 is a block diagram illustrating elements of an exemplary environment in which systems and methods for incident and event reporting may be implemented according to one embodiment of the present disclosure.

FIG. 4 is a flowchart illustrating an exemplary process for incident and event reporting according to one embodiment of the present disclosure.

FIG. 5 is a flowchart illustrating an exemplary process for receiving an incident or event report according to one embodiment of the present disclosure.

FIG. 6 is a flowchart illustrating an exemplary process for incident or event follow-up management according to one embodiment of the present disclosure.

FIG. 7 is a flowchart illustrating an exemplary process for validation and review of received incident or event reports according to one embodiment of the present disclosure.

FIG. 8 is a flowchart illustrating an exemplary process for analysis of incident or event records according to one embodiment of the present disclosure.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

While the exemplary aspects, embodiments, and/or configurations illustrated herein show the various components of the system collocated, certain components of the system can be located remotely, at distant portions of a distributed network, such as a Local-Area Network (LAN) and/or Wide-Area Network (WAN) such as the Internet, or within a dedicated system. Thus, it should be appreciated, that the components of the system can be combined in to one or more devices or collocated on a particular node of a distributed network, such as an analog and/or digital telecommunications network, a packet-switch network, or a circuit-switched network. It will be appreciated from the following description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, Non-Volatile Random-Access Memory (NVRAM), or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a Compact Disk Read-Only Memory (CD-ROM), any other optical medium, punch cards, paper tape, any other physical medium with patterns of holes, a Random-Access Memory (RAM), a Programmable Read-Only Memory (PROM), and Erasable Programable Read-Only Memory (EPROM), a Flash-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A “computer readable signal” medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, Radio Frequency (RF), etc., or any suitable combination of the foregoing.

The terms “determine,” “calculate,” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as Programmable Logic Device (PLD), Programmable Logic Array (PLA), Field Programmable Gate Array (FPGA), Programmable Array Logic (PAL), special purpose computer, any comparable means, or the like. In general, any device(s) or means capable of implementing the methodology illustrated herein can be used to implement the various aspects of this disclosure. Exemplary hardware that can be used for the disclosed embodiments, configurations, and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or Very Large-Scale Integration (VLSI) design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or Common Gateway Interface (CGI) script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

Various additional details of embodiments of the present disclosure will be described below with reference to the figures. While the flowcharts will be discussed and illustrated in relation to a particular sequence of events, it should be appreciated that changes, additions, and omissions to this sequence can occur without materially affecting the operation of the disclosed embodiments, configuration, and aspects.

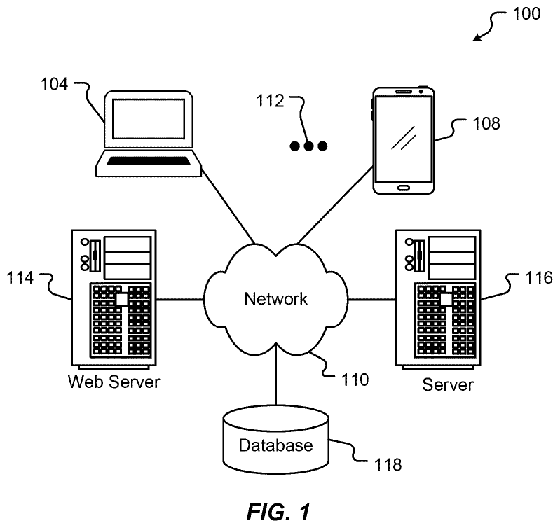

FIG. 1 is a block diagram illustrating elements of an exemplary computing environment in which embodiments of the present disclosure may be implemented. More specifically, this example illustrates a computing environment 100 that may function as the servers, user computers, or other systems provided and described herein. The environment 100 includes one or more user computers, or computing devices, such as a computing device 104, a communication device 108, and/or more 112. The computing devices 104, 108, 112 may include general purpose personal computers (including, merely by way of example, personal computers, and/or laptop computers running various versions of Microsoft Corp.'s Windows® and/or Apple Corp.'s Macintosh® operating systems) and/or workstation computers running any of a variety of commercially-available UNIX® or UNIX-like operating systems. These computing devices 104, 108, 112 may also have any of a variety of applications, including for example, database client and/or server applications, and web browser applications. Alternatively, the computing devices 104, 108, 112 may be any other electronic device, such as a thin-client computer, Internet-enabled mobile telephone, and/or personal digital assistant, capable of communicating via a network 110 and/or displaying and navigating web pages or other types of electronic documents. Although the exemplary computer environment 100 is shown with two computing devices, any number of user computers or computing devices may be supported.

Environment 100 further includes a network 110. The network 110 may can be any type of network familiar to those skilled in the art that can support data communications using any of a variety of commercially-available protocols, including without limitation Session Initiation Protocol (SIP), Transmission Control Protocol/Internet Protocol (TCP/IP), Systems Network Architecture (SNA), Internetwork Packet Exchange (IPX), AppleTalk, and the like. Merely by way of example, the network 110 maybe a Local Area Network (LAN), such as an Ethernet network, a Token-Ring network and/or the like; a wide-area network; a virtual network, including without limitation a Virtual Private Network (VPN); the Internet; an intranet; an extranet; a Public Switched Telephone Network (PSTN); an infra-red network; a wireless network (e.g., a network operating under any of the IEEE 802.9 suite of protocols, the Bluetooth® protocol known in the art, and/or any other wireless protocol); and/or any combination of these and/or other networks.

The system may also include one or more servers 114, 116. In this example, server 114 is shown as a web server and server 116 is shown as an application server. The web server 114, which may be used to process requests for web pages or other electronic documents from computing devices 104, 108, 112. The web server 114 can be running an operating system including any of those discussed above, as well as any commercially-available server operating systems. The web server 114 can also run a variety of server applications, including SIP servers, HyperText Transfer Protocol (secure) (HTTP(s)) servers, FTP servers, CGI servers, database servers, Java servers, and the like. In some instances, the web server 114 may publish operations available operations as one or more web services.

The environment 100 may also include one or more file and or/application servers 116, which can, in addition to an operating system, include one or more applications accessible by a client running on one or more of the computing devices 104, 108, 112. The server(s) 116 and/or 114 may be one or more general purpose computers capable of executing programs or scripts in response to the computing devices 104, 108, 112. As one example, the server 116, 114 may execute one or more web applications. The web application may be implemented as one or more scripts or programs written in any programming language, such as Java™, C, C#®, or C++, and/or any scripting language, such as Perl, Python, or Tool Command Language (TCL), as well as combinations of any programming/scripting languages. The application server(s) 116 may also include database servers, including without limitation those commercially available from Oracle®, Microsoft®, Sybase®, IBM® and the like, which can process requests from database clients running on a computing device 104, 108, 112.

The web pages created by the server 114 and/or 116 may be forwarded to a computing device 104, 108, 112 via a web (file) server 114, 116. Similarly, the web server 114 may be able to receive web page requests, web services invocations, and/or input data from a computing device 104, 108, 112 (e.g., a user computer, etc.) and can forward the web page requests and/or input data to the web (application) server 116. In further embodiments, the server 116 may function as a file server. Although for ease of description, FIG. 1 illustrates a separate web server 114 and file/application server 116, those skilled in the art will recognize that the functions described with respect to servers 114, 116 may be performed by a single server and/or a plurality of specialized servers, depending on implementation-specific needs and parameters. The computer systems 104, 108, 112, web (file) server 114 and/or web (application) server 116 may function as the system, devices, or components described herein.

The environment 100 may also include a database 118. The database 118 may reside in a variety of locations. By way of example, database 118 may reside on a storage medium local to (and/or resident in) one or more of the computers 104, 108, 112, 114, 116. Alternatively, it may be remote from any or all of the computers 104, 108, 112, 114, 116, and in communication (e.g., via the network 110) with one or more of these. The database 118 may reside in a Storage-Area Network (SAN) familiar to those skilled in the art. Similarly, any necessary files for performing the functions attributed to the computers 104, 108, 112, 114, 116 may be stored locally on the respective computer and/or remotely, as appropriate. The database 118 may be a relational database, such as Oracle 20i®, that is adapted to store, update, and retrieve data in response to Structured Query Language (SQL) formatted commands.

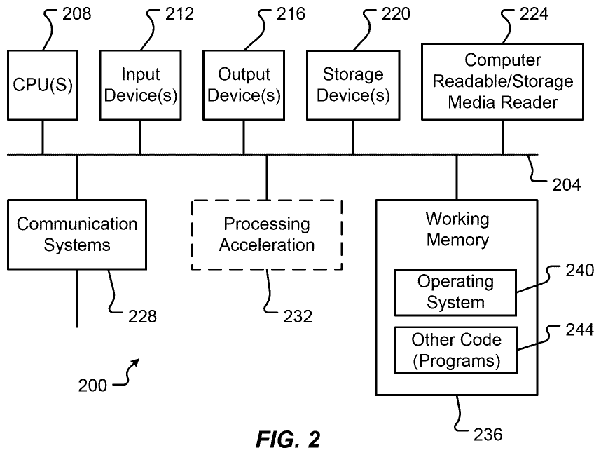

FIG. 2 is a block diagram illustrating elements of an exemplary computing device in which embodiments of the present disclosure may be implemented. More specifically, this example illustrates one embodiment of a computer system 200 upon which the servers, user computers, computing devices, or other systems or components described above may be deployed or executed. The computer system 200 is shown comprising hardware elements that may be electrically coupled via a bus 204. The hardware elements may include one or more Central Processing Units (CPUs) 208; one or more input devices 212 (e.g., a mouse, a keyboard, etc.); and one or more output devices 216 (e.g., a display device, a printer, etc.). The computer system 200 may also include one or more storage devices 220. By way of example, storage device(s) 220 may be disk drives, optical storage devices, solid-state storage devices such as a Random-Access Memory (RAM) and/or a Read-Only Memory (ROM), which can be programmable, flash-updateable and/or the like.

The computer system 200 may additionally include a computer-readable storage media reader 224; a communications system 228 (e.g., a modem, a network card (wireless or wired), an infra-red communication device, etc.); and working memory 236, which may include RAM and ROM devices as described above. The computer system 200 may also include a processing acceleration unit 232, which can include a Digital Signal Processor (DSP), a special-purpose processor, and/or the like.

The computer-readable storage media reader 224 can further be connected to a computer-readable storage medium, together (and, optionally, in combination with storage device(s) 220) comprehensively representing remote, local, fixed, and/or removable storage devices plus storage media for temporarily and/or more permanently containing computer-readable information. The communications system 228 may permit data to be exchanged with a network and/or any other computer described above with respect to the computer environments described herein. Moreover, as disclosed herein, the term “storage medium” may represent one or more devices for storing data, including ROM, RAM, magnetic RAM, core memory, magnetic disk storage mediums, optical storage mediums, flash memory devices and/or other machine-readable mediums for storing information.

The computer system 200 may also comprise software elements, shown as being currently located within a working memory 236, including an operating system 240 and/or other code 244. It should be appreciated that alternate embodiments of a computer system 200 may have numerous variations from that described above. For example, customized hardware might also be used and/or particular elements might be implemented in hardware, software (including portable software, such as applets), or both. Further, connection to other computing devices such as network input/output devices may be employed.

Examples of the processors 208 as described herein may include, but are not limited to, at least one of Qualcomm® Snapdragon® 800 and 801, Qualcomm® Snapdragon® 620 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

Embodiments of the present disclosure are directed to healthcare incident and event reporting and management. According to one embodiment, healthcare incident and event reporting and management can be implemented as a cloud-based service and provided to clients, such as healthcare providers, for example, over the Internet and/or other communications networks. Generally speaking, and as will be described in greater detail below, incident and event reporting and management can include reporting, follow-up management, and post-event analysis to help healthcare providers control and reduce the number of harmful incidents and events.

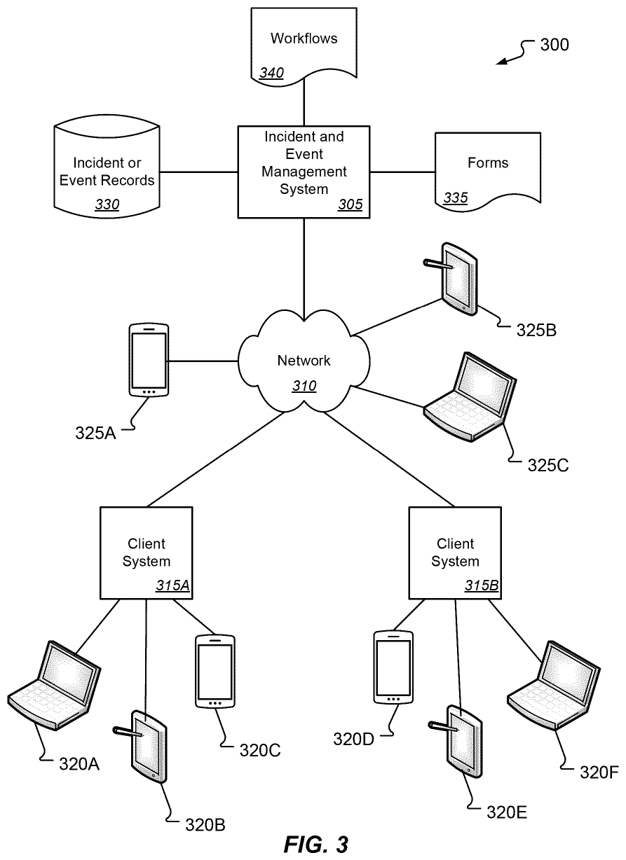

FIG. 3 is a block diagram illustrating elements of an exemplary environment in which systems and methods for incident and event reporting may be implemented according to one embodiment of the present disclosure. As illustrated in this example, an environment 300 for incident and event reporting can comprise an incident and event management system 305 implemented on any one or more servers and/or other computing devices as described above. The incident and event management system 305 can be communicatively coupled with one or more wired and/or wireless local and/or wide area networks 310 including, but not limited to, the Internet and/or any other communications network(s) such as described above.

The environment 300 can further comprise one or more client systems 315A and 315B also communicatively coupled with the network(s). The client system(s) 315A and 315B can comprise any one or more servers and/or other computing devices as described above installed, for example, in a healthcare provider facility such as a hospital, clinic, urgent care facility, assisted living facility, doctor's office, etc. Users within such facilities can connect with the client system(s) via one or more wired and/or wireless networks (not shown here) using any of a variety of user devices 320A-320F such as laptop computers, tablets, smart phones, or other devices as described above. Through these user devices 320A-320F, and the client system(s) 315A and 315B, the users can also access and interact with the incident and event management system 305. Additionally, or alternatively, users of user devices 325A-325C can access the incident and event management system 305 via the Internet or other network(s) 310 without first accessing or passing through a client system 315A or 315B.

Generally speaking, the incident and event reporting system 305 can perform a set of functions or services provided to users of client systems 315A and 315B and/or user devices 320A-320F and 325A-325C in a Software-as-a-Service (SaaS) model. The client systems 315A and 315B and user devices 320A-320F and 325A-325C can provide to the incident and event reporting system 305 incident or event data to be maintained by the incident and event management system 305 in a set of incident or event records 330, e.g., in a database or other repository. The services provided by the incident and event management system 305 can include, but are not limited to, incident and/or event reporting, follow-up management, root cause analysis, peer review, mortality review, analytics, and drill-through reporting. Any or all of these services can be customized to a particular client organization or user. Details of exemplary processes for incident and event report as can be performed by the incident and event management system 305 will be described below.

According to one embodiment, the incident and event management system 305 can be further adapted to provide additional services and/or solutions. That is, while described herein with reference to providing an incident or event reporting service, additional services including, but not limited to, performance monitoring and improvement, compliance monitoring and improvement, risk management, various administrative services, etc., can be provided by the incident and event management system 305. In such cases, the forms 335 can be adapted to collect the data appropriate and relevant to that service. Similarly, the workflows 340 can be adapted to perform actions appropriate to processing the collected data to provide the specific service. Users of the client system 315A or 315B to which such services are provided can access the services based on their roles or other permissions. According to one embodiment, these roles or permissions can be different between the different services provided.

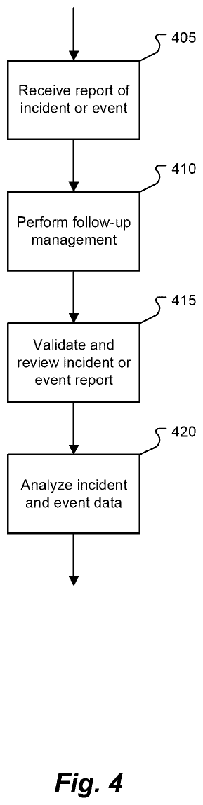

FIG. 4 is a flowchart illustrating an exemplary process for incident and event reporting according to one embodiment of the present disclosure. As illustrated in this example, incident and event reporting and management as can be performed by a system such as the incident and event management system 305 described above can begin with receiving 405 an incident or event report from a client system 305 and/or user device 320A-320F and 325A-325C. This report can be received though a graphical and/or textual user interface such as a web page, for example, presented by the incident and event management system 305 to a client system 315A or 315B and/or user device 320A-320F and 325A-325C and saved by the incident and event management system 305 in one or more event and incident records 330. Such a web page or other user interface can comprise one or more forms 335 which may be customized to a particular client or user, e.g., a hospital, clinic, urgent care facility, assisted living facility, doctor's office, etc., to track the data that's important to that client or user making it easy for users to report incidents quickly in a secure manner. For example, the forms can be adapted to include a particular user or client's logo, us customized icons and/or color schemes, etc. Such forms 335 can be used to provide a custom “landing page” as well and provide a unique but uniform view to different clients. Forms 335 can be toggled on or off depending upon a specific client's implementation and the types of forms used for that implementation.

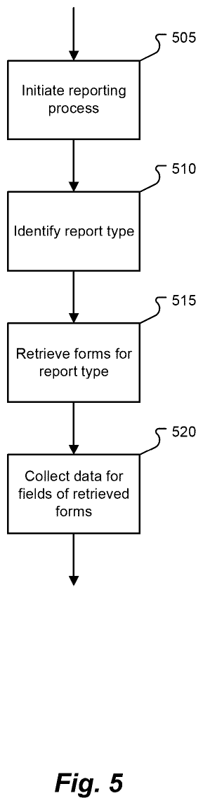

In other cases, report data may be received from a client system 315A or 315B through an Application Program Interface (API) or through other automatic processes. For example, a client system 315A can, through the API provided by the incident and event management system 305, submit incident and event data to be saved as an incident and event record 330. In another example, the incident and event management system 305 can periodically, or on demand, crawl one or more client systems 315A and/or 315B and scrape or collect incident and event data to be saved as an incident and event record 330. In either cases, the incident and event management system 305 can then make the collected reports saved as incident or event records 330 available to various types of client systems 315A and 315B and user devices 320A-320F and 325A-325C and provide interoperability with such other systems, e.g., EHR and physician rosters, making it easy to seamlessly pull data for faster reporting. Additional details of an exemplary process for receiving an incident or event report will be described below with reference to FIG. 5.

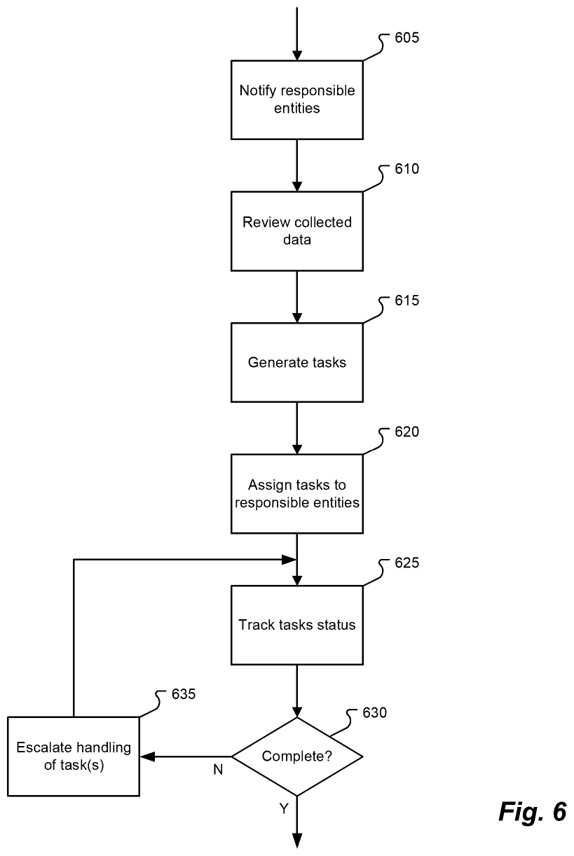

Follow-up management can be performed 410 by the incident and event management system 305. More specifically, after an incident or event is reported, the information obtained through the reporting form 335 and saved as an incident and event record 330 can be reviewed and a follow-up action plan can be formed and initiated accordingly. This review can be conducted by a human user, an automated process such as workflow 340, or a combination of a human user assisted or guided by an automated process. For example, when an event is initially reported, a reviewer can be informed, e.g., by an automatic email notification and/or an alert on an application dashboard. The dashboard can be presented using one or more forms 335 which, as noted above, can be customized for the client to which they are presented. Such a real-time dashboard presented in a user interface such as a web page, for example, by the incident and event management system 305 can give the reviewer a list of any events reported as well as their current status. An event can be automatically “flagged” when submission based on certain information provided in the event report. After examining the event report, the reviewer can then assign follow-up tasks to team members. A centralized channel for team communication can help to eliminate knowledge gaps that occur when information is exchanged via email chains or external discussions. Built-in escalation processes can help to keep team members on track and informed about next steps. Additional details of an exemplary process for follow-up management will be described below with reference to FIG. 6.

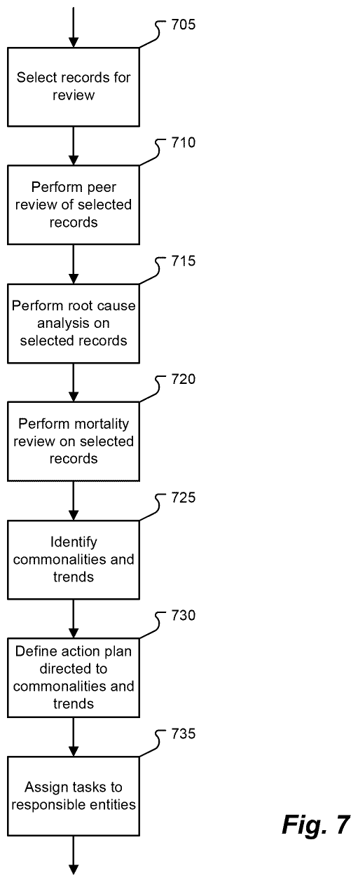

The incident and event management system 305 can also perform 415 validation and review of received incident or event reports saved as incident or event records 330. After reporting and reviewing, follow-up can be performed in order to track trends over time and understand how to prevent similar events from occurring in the future. This validation and review can include a peer review. Peer review can be conducted, for example, within a central dashboard presented in one or more webpages or other user interface(s) provided by the incident and event management system 305. Leadership teams can decide which events to peer review and invite team members to complete the review process. Documentation for performing the peer review can be easily accessed in a central location, e.g., the presented dashboard, for faster and more productive follow-up. Additional details of an exemplary process for validation and review of received incident or event reports will be described below with reference to FIG. 7.

The validation and review processes can also include performing a root cause analysis, i.e., systematic processes for identifying the root cause of an event. A root cause analysis process can comprise one or more automated processes, e.g., workflows 340 etc., to guide users step-by-step through identifying the root causes of the event. Through these processes, the causes can be identified in a simple, user-friendly way, and the event can be clearly described. Next, the causes can be specified on multiple levels to discover the root cause. The root causes bring valuable insights for quality-of-care recommendations.

The validation and review processes can also include performing a mortality review to identify, review, and implement action plans for eliminating and preventing mortality events. According to one embodiment, mortality reviews can be accessed through one or more webpages or other interface(s) provided by the incident and event management system 305, e.g., through the central dashboard, allowing care teams to proactively identify, review, and implement action plans to prevent mortality events.

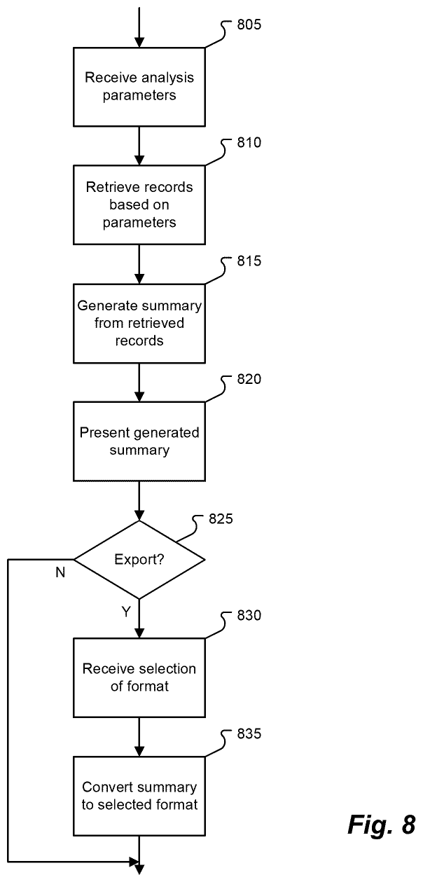

The incident and event data can be analyzed 420, e.g., periodically or upon request. Analyzing the incident and event data can comprise presenting in one or more webpages or other interface(s) provided by the incident and event management system 305 custom dashboards and analytics allowing care teams to gain a deeper understanding of what is going on in their organization and see exactly where improvements are necessary. The provided data analysis with drill-through reporting allows organizations to increase their response rates, make more informed decisions, implement action plans, and put procedures in place to improve patient outcomes in the future. Such dashboards can be personalized per client, per department, etc. and can provide various types of charts including, but not limited to, line, bar, and pie diagrams and can be exportable to different formats including, but not limited to, Word, Excel, PDF, CSV, and XML, etc. The analysis can also provide dynamic, color coordinated work lists, easy searching, grouping, and filtering of data with drill-through reporting and calculated turn-around times. Additional details of an exemplary process for analysis of incident or event records will be described below with reference to FIG. 8.

FIG. 5 is a flowchart illustrating an exemplary process for receiving an incident or event report according to one embodiment of the present disclosure. As illustrated in this example, receiving the report of the incident or event can comprise initiating 505 a reporting process with a user device though a communications network, e.g., by the user device automatically, or in response to a user action, generating a report request. In some cases, the request can identify the type of report to be made. A report type for the reporting process can be identified 510, e.g., based on the request from the user device, by querying the user device, or through other means.

A predefined form can be retrieved 515 for the report based on the identified report type and data for the report of the incident or event can be collected 520 from the user device through the communications network based on fields in the predefined form. Collecting 520 the data for each of the fields can comprise receiving input from a user and/or automatically collecting the information from the client system and/or user device. According to one embodiment, a client system can utilize a form's “question tags” by appending the value for the tag to the form's URL or otherwise passing the value to the form 335. When that form 335 is rendered or otherwise utilized, the value for the field associated with the question tag is then pre-populated in that field on the form. The user can include these values pre-populated by the client system with other values that the user manually inputs prior to submitting a form. Additionally, or alternatively, the question tags and the values for those question tags may be used to search, sort, or otherwise manage incident or event records.

According to one embodiment, one or more of the fields can be tagged identifying it as a commonly occurring reporting field, e.g., patient's name, date of incident or event, etc., across different forms. In such cases, the reporting fields and the collected data values for those fields can be used to aggregate data across multiple forms. More specifically, various reporting fields can be predefined by name, e.g., a “Name” reporting field, a “Date” reporting field , an “Event Type” reporting field, etc. Any of these reporting fields can be associated with a particular field in a form 335. When that form 335 is utilized by a client system 315A and a value for the field us entered or obtained, that value can be assigned to the associated reporting field for that form 335. Additionally, or alternatively, the reporting fields and the values for those reporting fields may be used to search, sort, or otherwise manage incident or event records.

FIG. 6 is a flowchart illustrating an exemplary process for incident or event follow-up management according to one embodiment of the present disclosure. As illustrated in this example, performing follow-up management on the report of the incident or event can comprise notifying 605 a responsible entity of the report of the incident or event. For example, the responsible entity may be a human reviewer and the notification can comprise an automatically generated email or other electronic communications. The incident or event data stored in the electronic incident or event record can then be reviewed 610 by the responsible entity

One or more tasks for addressing the incident or event can be generated 615 based on reviewing the incident or event data. For example, the tasks can comprise a corrective action to address the reported incident, a preventative action to reduce the likelihood of a similar incident of event from occurring again, or another action. The one or more tasks can then be assigned 620 to one or more responsible entities. The responsible entities can comprise one or more of a human user, an automated process such as a workflow, or a combination of a human user assisted or guided by an automated process. The tasks can be assigned and/or presented based on one or more customizable forms as described above. User access to view various tasks assigned to different entities can be based on the user's role and/or permissions. For example, the tasks can be added to a worklist. Through the worklist, reviewers can see how many forms have been assigned to follow-up by an entity. Through the worklist, the reviewer can select which of the reportable fields, i.e., the tag fields as described above, from the form should appear in the worklist.

Performing follow-up management on the report of the incident or event can further comprise tracking 625 a status of each of the one or more tasks and determining whether any of the one or more tasks remain incomplete after a predetermined period of time assigned for completion of the task. In response to determining a task of the one or more task remains incomplete after the predetermined period of time for completion of the task, an escalation for handling the task can be initiated 635. The escalation for handling the task can comprise one or more automatic, manual, or combination of automatic and manual actions including, but not limited to, requesting a status or other information regarding the task, reassigning the task, updating the time for completion, etc.

FIG. 7 is a flowchart illustrating an exemplary process for validation and review of received incident or event reports according to one embodiment of the present disclosure. As illustrated in this example, validating and reviewing the electronic incident or event record can comprise selecting 710 one or more stored electronic incident or event records and performing one or more reviews or analyses on the selected one or more stored electronic records. For example, the review or analysis can comprise performing 710 a peer review of one or more records. Additionally, or alternatively, the review or analysis can comprise performing 715 a root cause analysis on one or more records. In yet another example, the review or analysis can additionally, or alternatively, comprise performing 720 a mortality review on the selected one or more records. It should be noted that any one such review or analysis as well as any combination of some or all of these reviews or analyses can be performed. According to one embodiment, the different reviews or analyses can be performed by different entities, e.g., human user, an automated process such as a workflow, or a combination of a human user assisted or guided by an automated process.

One or more commonalities or trends in incidents or events represented by the selected one or more records can be identified 725 based on performing the review or analysis on the selected one or more stored electronic records. An action plan directed to the identified one or more commonalities or trends can then be defined 730. The action plan can define one or more tasks to be performed, e.g., tasks to improve a condition, change a practice or procedure, implement a new practice or procedure, etc. in order to correct the identified commonalities or trends. The one or more tasks defined in the action plan can then be assigned 735 to one or more responsible entities, i.e., a human user, an automated process such as a workflow, or a combination of a human user assisted or guided by an automated process.

FIG. 8 is a flowchart illustrating an exemplary process for analysis of incident or event records according to one embodiment of the present disclosure. As illustrated in this example, analyzing the incident or event based on the incident or event record can comprise receiving 805, from a user device, through a communications network, one or more analysis parameters and retrieving 810 one or more stored electronic incident or event records based on the received analysis parameters. For example, the parameters can define a range of dates in which incidents or events occurred, a geographic area from which the incidents or events were reported, a particular facility from which the incidents or events were reported, etc. According to one embodiment, the parameters can be based on one or more “reportable fields” used to filter data presented.

A summary of incidents or events represented by the retrieved one or more stored electronic incident or event records can be generated 815 and the generated summary of the incidents of events represented by the retrieved one or more stored electronic records can be presented 820 to the user device through the communications network. For example, the generated summary can be presented in the form of a graphical and/or textual user interface including a dashboard. As noted above, such dashboards can be personalized per client, per department, etc. and can provide various types of charts including, but not limited to, line, bar, and pie diagrams.

A determination 825 can be made as to whether to export the generated summary. For example, this determination can be based on receiving 830, from the client device, through the communications network, a request with a selection of an export format for the generated summary of the incidents of events represented by the retrieved one or more stored electronic records. In other cases, the indication to export and the selected format can be predetermined and made automatically. In either case, the generated summary of the incidents of events represented by the retrieved one or more stored electronic records can be converted 835 to the export format. A file containing the summary in the export format can then be saved and/or transmitted to various users.

The present disclosure, in various aspects, embodiments, and/or configurations, includes components, methods, processes, systems, and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for incident or event reporting, the method comprising:
receiving, by an incident and event management system, a report of an incident or event related to the delivery of healthcare services, the report of the incident or event comprising incident or event data describing the incident or event;
performing, by the incident and event management system, follow-up management on the report of the incident or event related to the delivery of healthcare services based on an electronic incident or event record storing the incident or event data;
validating and reviewing, by the incident and event management system, the electronic incident or event record; and
analyzing, by the incident and event management system, the incident or event based on the incident or event record.

2. The method of claim 1, wherein receiving the report of the incident or event comprises:
initiating a reporting process with a user device though a communications network;
identifying a report type for the reporting process based on a request from the user device;
retrieving a predefined form for the report based on the identified report type; and
collecting, from the user device through the communications network, data for the report of the incident or event based on fields in the predefined form.

3. The method of claim 1, wherein performing follow-up management on the report of the incident or event comprises:
notifying a responsible entity of the report of the incident or event;
reviewing the incident or event data stored in the electronic incident or event record;
generating one or more tasks for addressing the incident or event based on reviewing the incident or event data; and
assigning the one or more tasks to one or more responsible entities.

4. The method of claim 3, wherein performing follow-up management on the report of the incident or event further comprises:
tracking a status of each of the one or more tasks;
determining whether any of the one or more tasks remain incomplete after a predetermined period of time assigned for completion of the task; and
in response to determining a task of the one or more task remains incomplete after the predetermined period of time for completion of the task, initiating an escalation for handling the task.

5. The method of claim 1, wherein validating and reviewing the electronic incident or event record comprises:
selecting one or more stored electronic incident or event records;
performing at least one review or analysis on the selected one or more stored electronic records;
identifying one or more commonalities or trends in incidents or events represented by the selected one or more records based on performing the at least one review or analysis on the selected one or more stored electronic records;
defining an action plan directed to the identified one or more commonalities or trends, the action plan defining one or more tasks to be performed; and
assigning the one or more tasks defined in the action plan to one or more responsible entities.

6. The method of claim 5, wherein the at least one review or analysis comprises a peer review of the selected one or more records.

7. The method of claim 5, wherein the at least one review or analysis comprises a root cause analysis on the selected one or more records.

8. The method of claim 5, wherein the at least one review or analysis comprises a mortality review on the selected one or more records.

9. The method of claim 1, wherein analyzing the incident or event based on the incident or event record comprises:
receiving, from a user device, through a communications network, one or more analysis parameters;
retrieving one or more stored electronic incident or event records based on the received analysis parameters;
generating a summary of incidents or events represented by the retrieved one or more stored electronic incident or event records; and
presenting, to the user device, through the communications network, the generated summary of the incidents of events represented by the retrieved one or more stored electronic records.

10. The method of claim 9, wherein the incident or event based on the incident or event record further comprises:
receiving, from the client device, through the communications network, a selection of an export format for the generated summary of the incidents of events represented by the retrieved one or more stored electronic records; and
converting the generated summary of the incidents of events represented by the retrieved one or more stored electronic records to the export format.

11. A system comprising:
a processor; and
a memory coupled with and readable by the processor and storing therein a set of instructions which, when executed by the processor, causes the processor to manage reporting of incidents or events by:
receiving a report of an incident or event related to the delivery of healthcare services, the report of the incident or event comprising incident or event data describing the incident or event;
performing follow-up management on the report of the incident or event related to the delivery of healthcare services based on an electronic incident or event record storing the incident or event data;
validating and reviewing the electronic incident or event record; and
analyzing the incident or event based on the incident or event record.

12. The system of claim 11, wherein receiving the report of the incident or event comprises:
initiating a reporting process with a user device though a communications network;
identifying a report type for the reporting process based on a request from the user device;
retrieving a predefined form for the report based on the identified report type; and
collecting, from the user device through the communications network, data for the report of the incident or event based on fields in the predefined form.

13. The system of claim 11, wherein performing follow-up management on the report of the incident or event comprises:
notifying a responsible entity of the report of the incident or event;
reviewing the incident or event data stored in the electronic incident or event record;
generating one or more tasks for addressing the incident or event based on reviewing the incident or event data;
assigning the one or more tasks to one or more responsible entities;
tracking a status of each of the one or more tasks;
determining whether any of the one or more tasks remain incomplete after a predetermined period of time assigned for completion of the task; and
in response to determining a task of the one or more task remains incomplete after the predetermined period of time for completion of the task, initiating an escalation for handling the task.

14. The system of claim 11, wherein validating and reviewing the electronic incident or event record comprises:
selecting one or more stored electronic incident or event records;
performing at least one review or analysis on the selected one or more stored electronic records, wherein the at least one review or analysis comprises at least one of a peer review of the selected one or more records, a root cause analysis on the selected one or more records, or a mortality review on the selected one or more records;
identifying one or more commonalities or trends in incidents or events represented by the selected one or more records based on performing the at least one review or analysis on the selected one or more stored electronic records;
defining an action plan directed to the identified one or more commonalities or trends, the action plan defining one or more tasks to be performed; and
assigning the one or more tasks defined in the action plan to one or more responsible entities.

15. The system of claim 1, wherein analyzing the incident or event based on the incident or event record comprises:
receiving, from a user device, through a communications network, one or more analysis parameters;
retrieving one or more stored electronic incident or event records based on the received analysis parameters;
generating a summary of incidents or events represented by the retrieved one or more stored electronic incident or event records;
presenting, to the user device, through the communications network, the generated summary of the incidents of events represented by the retrieved one or more stored electronic records;
receiving, from the client device, through the communications network, a selection of an export format for the generated summary of the incidents of events represented by the retrieved one or more stored electronic records; and
converting the generated summary of the incidents of events represented by the retrieved one or more stored electronic records to the export format.

16. A non-transitory, computer-readable medium comprising a set of instructions stored therein which, when executed by a processor, causes the processor to manage reporting of incidents or events by:
receiving a report of an incident or event related to the delivery of healthcare services, the report of the incident or event comprising incident or event data describing the incident or event;
performing follow-up management on the report of the incident or event related to the delivery of healthcare services based on an electronic incident or event record storing the incident or event data;
validating and reviewing the electronic incident or event record; and
analyzing the incident or event based on the incident or event record.

17. The non-transitory, computer-readable medium of claim 16, wherein receiving the report of the incident or event comprises:
initiating a reporting process with a user device though a communications network;
identifying a report type for the reporting process based on a request from the user device;
retrieving a predefined form for the report based on the identified report type; and
collecting, from the user device through the communications network, data for the report of the incident or event based on fields in the predefined form.

18. The non-transitory, computer-readable medium of claim 16, wherein performing follow-up management on the report of the incident or event comprises:
notifying a responsible entity of the report of the incident or event;
reviewing the incident or event data stored in the electronic incident or event record;
generating one or more tasks for addressing the incident or event based on reviewing the incident or event data;
assigning the one or more tasks to one or more responsible entities;
tracking a status of each of the one or more tasks;
determining whether any of the one or more tasks remain incomplete after a predetermined period of time assigned for completion of the task; and
in response to determining a task of the one or more task remains incomplete after the predetermined period of time for completion of the task, initiating an escalation for handling the task.

19. The non-transitory, computer-readable medium of claim 16, wherein validating and reviewing the electronic incident or event record comprises:
selecting one or more stored electronic incident or event records;
performing at least one review or analysis on the selected one or more stored electronic records, wherein the at least one review or analysis comprises at least one of a peer review of the selected one or more records, a root cause analysis on the selected one or more records, or a mortality review on the selected one or more records;
identifying one or more commonalities or trends in incidents or events represented by the selected one or more records based on performing the at least one review or analysis on the selected one or more stored electronic records;
defining an action plan directed to the identified one or more commonalities or trends, the action plan defining one or more tasks to be performed; and
assigning the one or more tasks defined in the action plan to one or more responsible entities.

20. The non-transitory, computer-readable medium of claim 16, wherein analyzing the incident or event based on the incident or event record comprises:
receiving, from a user device, through a communications network, one or more analysis parameters;
retrieving one or more stored electronic incident or event records based on the received analysis parameters;
generating a summary of incidents or events represented by the retrieved one or more stored electronic incident or event records;
presenting, to the user device, through the communications network, the generated summary of the incidents of events represented by the retrieved one or more stored electronic records;
receiving, from the client device, through the communications network, a selection of an export format for the generated summary of the incidents of events represented by the retrieved one or more stored electronic records; and
converting the generated summary of the incidents of events represented by the retrieved one or more stored electronic records to the export format.
